(19) United States
(12) Patent Application Publication
(10) Pub. No.: US 2004/0071679 A1
(43) Pub. Date: Apr. 15, 2004

(54) Identifying and treating vaginal infections

(76) Inventor: Claudio De Simone, Ardea (IT)

Correspondence Address: NIXON & VANDERHYE, PC, 1100 N GLEBE ROAD, 8TH FLOOR, ARLINGTON, VA, 22201-4714, US

(21) Appl. No.: 10/270,167

(22) Filed: Oct. 15, 2002

(51) Int. Cl. A61K 45/00

(52) U.S. Cl. 424/93.45

(57) ABSTRACT

The presence of a vaginal affection is determined in a subject using an identifiable indicator and, if the indication is positive, a gynecological composition comprising one or more Lactobacillus strains is administered locally in an amount effective to treat the vaginal affection. Also described are packages or kits including a probe or tool such as a glove bearing a reporter substance for indicating the presence of a vaginal infection packaged together with a gynecological composition for treating the vaginal infection, if it is found to be present.

The present invention relates to improving the health of women having vaginal affections and to procedures for determining the presence of a vaginal affection and, if present, topical use of a gynecological composition comprising Lactobacillus strains. In a preferred aspect the invention relates to a method for determining the presence of a vaginal infection in a subject using an identifiable indicator and, if the indication is positive, the use of a gynecological composition comprising one or more Lactobacillus strains administered locally in an amount effective to treat the vaginal infection. Also described are packages or kits including a probe or tool such as a glove bearing a reporter substance indicating the presence of an infection packaged together with a gynecological composition for treating the vaginal affections, if it is found to be present.

The expression vaginal affections as used herein includes vaginal infections such as bacterial vaginosis and allergic vaginitis. Presence of a vaginal affection may be indicated by change (increase) in the pH of vaginal fluid, a homogenous non-inflammatory vaginal secretion adhering to the vaginal walls, the presence of indicator cells or strong or bad odor. The gynecological compositions of the invention are also beneficial in the prevention or treatment of dyspareunia of which a vaginal infection is one of the causes.

Vaginal infections such as bacterial vaginosis, the consequence of a localized bacterial infection, and vaginitis associated with a local infection due to T. vaginalis or Candida especially Candida albicans may be detected by various indicators. The means to detect the presence of a vaginal infection are not specifically limited. Among those available are pH sensors, it being well known an increase in pH of the vaginal fluid to say 4.7 indicates the likely presence of a vaginal infection, while lower pH values of 4.0 to 4.4 are regarded as normal for most patients. On the other hand, an increase in pH is also an indication of a generic affection, as intended in the present invention. If the pH is lower than 4.7, there is no affection, and the compositions used in the invention may be applied for preventing an affection.

The probe, used to collect a sample of the vaginal fluid, may take any convenient shape such as an applicator, cotton swab or wand. A convenient, disposable single use probe such as a rubber or plastic examination glove, optionally provided with an indicator/reporter substance or substances thereon, may be used, although the indicator/reporter substance may form part of a separate device. Such products include disposable gynecological examination gloves bearing thereon a pH-sensitive test indicator are commercially available, such as CarePlan VpH gynecological examination gloves available from Selfcare Inc. Alternatively there is FemExam®, a combined pH and amines test card available from CooperSurgical, Inc. This is a credit-card size device having calorimetric test circular areas to distinguish pH 4.7 or greater as well as for the presence of volatile amines, both indicators of a bacterial vaginal infection. Using these cards a cotton-tipped swab or similar arrangement is used to collect then to apply a sample of vaginal fluid to the test areas. If present, elevated vaginal pH is noted and in a separate test area; the presence of volatile amines is also reported.

If the probe/self-examination device does not include a substance thereon that is self-readable, the package will also include means of assessing the results of (self) examination to indicate the presence, or not, of a bacterial infection. For example, if pH is the indicator to be detected/assessed the package will include an arrangement of multi-colored strips, bands or other indicia to compare the color generated by the signal substance when exposed to the sample of vaginal fluid to demonstrate to the user/patient the results of examination and if therapeutic countermeasures are indicated. A rise in pH is indicated by an appropriate color-coded swatch or calibrated comparison chart above a predetermined color, hence pH value, then therapeutic procedures are in order.

Using an indicator bearing examination glove of the type described the presence of pH modification is (self) assessed by donning a disposable examination glove, the finger portion(s) provided with an indicator substance or substances, contacting a gloved finger with vaginal fluid to collect a sample and come into contact with the indicator substance, then comparing the color generated from the thus-contacted finger portion with an interpretation chart, if needed for the indicator employed. Again, using pH as the variable to be tracked, the examination probe is provided with a pH-indication color-changing substance which after contact with fluid shows an identifiable change in color. Color change is assessed and a value assigned against multiple color interpretation swatches or series of colors associated with a given pH value or range in values. Typical values range from 4.0 to 4.4 for normal pH with increasing pH values in intervals of 0.2 or 0.3, such as intervals of 4.7, 5.0, 5.3, 5.5 and 5.8. A pH greater than 4.7 is generally regarded by clinicians to indicate the presence of an infection, alerts the user therapeutic intervention is in order. On the other hand, if the pH is below 4.7, no affection is present, and the compositions herein described are used to prevent an affection.

Once an abnormality or change in condition is observed and it is apparent therapy is advisable, treatment is instituted. Active ingredients to combat the infection may be selected from a wide range of lactic acid bacteria such as Lactobacillus acidophilus, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus jensenii, Lactobacillus leichmannii, Lactobacillus minutus, Lactobacillus plantarum, Lactobacillus rogosae, Lactobacillus salivarius, Lactobacillus brevis, Lactobacillus gasseri, Lactobacillus fermentum, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudo-catenulatum, Bifidobacterium pseudolongum, Streptococcus lactis, Streptococcus raffinolactis, Acidaminococcus fermenta, Cytophaga fermentans, Rhodoferax fennentans, Cellulomonas fermentans, Zymomonas mobilis, and Streptococcus thermophilus. Preferred, according to one embodiment of the invention, are those chosen from Lactobacillus brevis and Lactobacillus salivarius subs. salicinius species optionally used in combination with one or more species of lactobacilli selected from Lactobacillus salivarius subs. salivarius, Lactobacillus jensenii, Lactobacillus catenaforme, Lactobacillus minutus and Lactobacillus gasseri.

Preferably, the association of bacteria used in the pharmaceutical composition comprises or consists of Lactobacillus brevis, Lactobacillus salivarius subs. salicinius and Lactobacillus gasseri. Particular examples of lactobacilli to be used are Lactobacillus brevis ATCC 4006 and ATCC 14869, Lactobacillus salivarius subs. salicinius ATCC 11742 and Lactobacillus gasseri ATCC 9857.

Preferably, in the association of bacteria employed in accordance with the present invention the bacteria concentration is 10⁷ to 10¹³ CFU/g, more preferably 10⁸ to 10¹² CFU/g, most preferably more than 10⁹ to 10¹² CFU/g. Preferably, in the association of bacteria each species is present at a concentration of 10⁸ to 10¹² CFU/g. The bacterial cultures preferably are in a lyophilized form.

The lactobacilli-containing compositions used in the present invention may also be used to treat allergic vaginitis.

Another therapeutic product useful for treating vaginal conditions, particularly vaginal infections, is tea tree oil, a natural product extracted from Melaleuca Alternifolia leaves, an oil extracted by distillation containing tupinene-4-ol. Tea tree oil may be taken orally in a solubilized aqueous solution or applied topically or in a vaginal tablet when included in the appropriate formulating agent(s).

For practical use the compositions used in the invention, including gynecological, cosmetic and pharmaceutical compositions, are prepared in any convenient form for topical application such as in a liquid form, in the form of creams or ointments, or in a solid form, i.e. as pessaries or vaginal tablets, packets and the like. The compositions made in the form of vaginal tablets can be of a single layer or two or more layers having differentiated release times.

In one embodiment of the invention, the compositions of the present invention can be prepared in the form of tablets made up of two layers. Tea tree oil may also be included in one or both layers of the tablet. Such two layers, both containing a species of lactobacilli, bound with usual excipients and additives, can be arranged in such a manner that bacteria in the outer layer are released in a lapse of time of 10-25 minutes, about 15-20 minutes for example, whereas bacteria of the inner layer are released subsequently in a lapse of time of 25-50 minutes, about 30-40 minutes for example.

The compositions may also contain a buffering agent capable of maintaining an intervaginal pH stabilized in a range between 3 and 5.5 for some hours after administration. The buffering agent is a buffer system consisting of a weak acid selected from any pharmaceutially-acceptable inorganic or organic weak acid, such as boric acid, lactic acid, ascorbic acid, citric acid or acetic acid for example, in combination with the respective sodium salt or another pharmaceutically acceptable salt of the conjugated base of the weak acid used. Preferably, the pH is buffered in a range of 4.2 to 4.5 and preferably the buffer agent used is a buffer system made up of lactic acid and sodium lactate or ascorbic acid and sodium ascorbate. These and related compositions are described in DeSimone et al U.S. Pat. No. 6,277,370 the entire content of which is incorporated by reference.

In another preferred embodiment of the invention the pharmaceutical compositions used for therapy herein are described in DeSimone WO 00/78322 A2 and its counterpart U.S. application Ser. No. 10/024,199 filed Dec. 21, 2001, the entire content of which is hereby incorporated by reference. This embodiment employs a combination of lactic acid bacteria including (a) a first component consisting of at least one strain of an H₂O₂-producing lactic acid bacteria and (b) a second component consisting of a strain of arginine-utilizing bacteria.

The lactic acid bacteria in component (b) is the Lactobacillus brevis CD2 strain deposited under the Budapest Treaty under accession no. DSM 11988. The ratio of the amounts of bacteria in components (a): (b) will range from 100:1 to 1:100, preferably 1:5 to 5:1 and desirably in a substantially equal ratio of 1:1. Generally a unit dosage will contain 1×10² to 5×10¹¹ bacteria of component (a) and from 1×10² to 5×10¹¹ bacteria of component (b) with preferred amounts being 1×10⁹ bacteria for component (a) and 3×10⁹ bacteria of component (b).

A preferred combination of lactic acid bacteria contains (a) a first component consisting of at least one strain of H₂O₂-producing lactic acid bacteria, and (b) a second component consisting of at least one strain of arginine-utilizing lactic acid bacteria, where component (a) is selected from strains of the species Lactobacillus crispatus, Lactobacillus salivarius and Lactobacillus casei, and component (b) is selected from strains of the species Lactobacillus brevis, Lactobacillus gasseri and Lactobacillus fermentum, provided that when component (a) is Lactobacillus casei, component (b) is not Lactobacillus gasseri or Lactobacillus fennentum and when component (a) is Lactobacillus crispatus, component (b) is not Lactobacillus fennentum.

The composition may additionally include at least one other strain of lactic acid bacteria provided that when component (a) is Lactobacillus casei, the other strain is not Lactobacillus gasseri or Lactobacillus fermentum and when component (a) is Lactobacillus crispatus, and the other strain is not Lactobacillus fermentum. Additionally vitamins, quaternary ammonium bases, mineral salts, tea tree oil and antioxidant agents may also be present.

Particularly preferred compositions are when component (a) is Lactobacillus crispatus and component (b) is Lactobacillus brevis or when component (a) is Lactobacillus salivarius and component (b) is Lactobacillus brevis.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method of treating vaginal affections comprising the steps of:
(a) determining the presence of a vaginal affection, and, if present,
(b) topically applying to the affected area a composition comprising lactic acid bacteria.

2. A procedure for identifying and treating vaginal affections and/or vaginal dysmicrobism, comprising the steps of:
(a) securing a sample of vaginal fluid,
(b) exposing the collected fluid to an indicator signal substance,
(c) reading the exposed indicator signal substance and determining the predicted presence of a vaginal affection in said sample, and if an affection is present,
(d) applying to the area of affection a composition comprising lactic acid bacteria.

3. The procedure of claim 2, wherein the probe is an examination glove having a pH indicator signal substance thereon and, when the read pH value exceeds pH 4.7, conducting step (c).

4. The procedure of claim 2, wherein bacterial vaginosis is detected and treated.

5. The procedure of claim 2, wherein vaginitis is detected and treated.

6. The procedure of claim 2, wherein the composition applied in step (d) comprises lactic acid bacteria selected from the group consisting of Lactobacillus acidophilus, Lactobacillus buchneri, Lactobacillus casei, Lactobacillus catenaforme, Lactobacillus cellobiosus, Lactobacillus crispatus, Lactobacillus curvatus, Lactobacillus delbrueckii, Lactobacillus jensenii, Lactobacillus leichmannii, Lactobacillus minutus, Lactobacillus plantarum, Lactobacillus rogosae, Lactobacillus salivarius, Lactobacillus brevis, Lactobacillus gasseri, Lactobacillus fermentum, Bifidobacterium adolescentis, Bifidobacterium angulatum, Bifidobacterium bifidum, Bifidobacterium breve, Bifidobacterium catenulatum, Bifidobacterium dentium, Bifidobacterium eriksonii, Bifidobacterium infantis, Bifidobacterium longum, Bifidobacterium plantarum, Bifidobacterium pseudo-catenulatum, Bifidobacterium pseudolongum, Streptococcus lactis, Streptococcus raffinolactis, Acidaminococcus fermenta, Cytophaga fermentans, Rhodoferax fermentans, Cellulomonas fermentans, Zymomonas mobilis, and Streptococcus thermophilus.

7. The procedure of claim 6, wherein the lactic acid bacteria are selected from Lactobacillus brevis and Lactobacillus salivarius.

8. The procedure of claim 2, wherein the composition applied in step (d) comprises lactobacilli of the Lactobacillus brevis and Lactobacillus salivarius subs. salicinius species, in the form of pessaries or vaginal tablets comprising a layer of lactobacilli, in bound form, to control the release velocity of bacteria.

9. The procedure of claim 2, wherein the composition applied in step (d) comprises lactobacilli of the Lactobacillus brevis and Lactobacillus salivarius subs. salicinius species, in the form of pessaries or vaginal tablets comprising at least two layers, both layers containing said species of lactobacilli, in bound form, so that the release velocity of bacteria of the outermost layer is greater than the release velocity of bacteria of the innermost layer.

10. The procedure of claim 6, wherein said composition further comprises one or more species of lactobacilli selected from the group consisting of Lactobacillus salivarius subs. salivarius, Lactobacillus jensenii, Lactobacillus catenaforme, Lactobacillus minutus, Lactobacillus gasseri and Lactobacillus casei.

11. The procedure of claim 7, wherein said lactobacilli consist of the Lactobacillus brevis, Lactobacillus casei, Lactobacillus salivarius subs. salicinius and Lactobacillus gasseri species.

12. The procedure of claim 2, wherein said lactobacilli are present at a concentration of 10⁷ to 10¹³ CFU/g.

13. A method of treating allergic vaginitis comprising applying to the affected area an effective, allergy-relieving amount of a gynecological composition comprising lactic acid bacteria.

14. A method of prevention or treating dyspareunia comprising applying to the affected area a gynecological composition comprising lactic acid bacteria.

15. A method of treating vaginal affections comprising the steps of:
(a) determining the presence of a vaginal affection and, if present
(b) topically applying to the affected area a composition comprising tea oil.

16. A gynecological testing device comprising:
(a) a gynecological examination probe for securing a sample of vaginal fluid to be assessed;
(b) an indicator signal device indicating a pH of 4.7 or greater for identifying a vaginal affection when exposed to a sample of vaginal fluid;
(c) a gynecological composition qfor treating vaginal infections, and
(d) associated with the package, instructions for use of the probe, indicator signal device and composition to treat a vaginal infection.

17. The testing device of claim 16 in which the gynecological examination device is a glove.

18. The testing device of claim 16 in which the indicator signal substance is calorimetric.

19. A gynecological testing device comprising:
(a) a probe to collect a sample of vaginal fluid;
(b) a surface having thereon an indicator signal capable of indicating pH in excess of 4.7;
(c) a topically appliable antibacterial composition; and
(d) associated with the package, instructions for use of the probe and indicator signal to treat a vaginal infection.

20. The testing device of claim 19 in which the indicator signal is colorimetric.

21. A gynecological testing device comprising:
(a) a gynecological examination probe for securing a sample of vaginal fluid to be assessed;
(b) an indicator signal device indicating a pH of below 4.7;
(c) a gynecological composition for preventing vaginal infections, and
(d) associated with the package, instructions for use of the probe, indicator signal device and composition to prevent a vaginal infection.

22. The testing device of claim 21 in which the gynecological examination device is a glove.

23. The testing device of claim 21 in which the indicator signal substance is colorimetric.
